SEQUENCE LISTING (10 sequences)

SEQ ID NO: 1 - DNA, 69 bp, Artificial Sequence
  Synthetic construct
  misc_feature (25)..(27): n is a, c, g, or t
  misc_feature (30)..(33): n is a, c, g, or t
  misc_feature (36)..(39): n is a, c, g, or t
  misc_feature (42)..(44): n is a, c, g, or t
caagcagaagacggcatacgagatnnnatnnnncannnncgnnntggtcatgtggagacgctgggacag

SEQ ID NO: 2 - DNA, 24 bp, Artificial Sequence
  Synthetic construct
caagcagaagacggcatacgagat

SEQ ID NO: 3 - DNA, 25 bp, Artificial Sequence
  Synthetic construct
ctgtcccagcgtctccacatgacca

SEQ ID NO: 4 - DNA, 33 bp, Artificial Sequence
  Synthetic construct
gctgggacaggtcacttttcgtgcgccgcttca

SEQ ID NO: 5 - DNA, 25 bp, Artificial Sequence
  Synthetic construct
acactctttccctacattaactgca

SEQ ID NO: 6 - DNA, 18 bp, Artificial Sequence
  Synthetic construct
tctccgagcccacgagac

SEQ ID NO: 7 - DNA, 28 bp, Artificial Sequence
  Synthetic construct
tgggctcggagatgtgtataagagacag

SEQ ID NO: 8 - DNA, 24 bp, Artificial Sequence
  Synthetic construct
caagcagaagacggcatacgagat

SEQ ID NO: 9 - DNA, 20 bp, Artificial Sequence
  Synthetic construct
tcgtcggcagcgtcagatgt

SEQ ID NO: 10 - DNA, 56 bp, Artificial Sequence
  Synthetic construct
  misc_feature (19)..(21): n is a, c, g, or t
  misc_feature (24)..(27): n is a, c, g, or t
  misc_feature (30)..(33): n is a, c, g, or t
  misc_feature (36)..(38): n is a, c, g, or t
gaagacggcatacgagatnnnatnnnncannnncgnnngtctcgtgggctcggaga

(19) United States
(12) Patent Application Publication
(10) Pub. No.: US 2024/0263227 A1
(43) Pub. Date: Aug. 8, 2024

(54) METHODS OF BARCODING NUCLEIC ACID FOR DETECTION AND SEQUENCING

(71) Applicant: Universal Sequencing Technology Corporation, Canton, MA (US)

(72) Inventors: Zhoutao Chen, Carlsbad, CA (US); Devin Porter, Carlsbad, CA (US); Haibiao Gong, San Diego, CA (US)

(73) Assignee: Universal Sequencing Technology Corporation, Canton, MA (US)

(21) Appl. No.: 17/904,394

(22) PCT Filed: Feb. 17, 2021
(86) PCT No.: PCT/US2021/018423

§ 371 Date: Aug. 17, 2022

Related U.S. Application Data
(60) Provisional application No. 62/977,618, filed on Feb. 17, 2020.

(51) Int. Cl. C12Q 1/6869; C12Q 1/6806; C12Q 1/6841; C12Q 1/6844

(52) U.S. Cl. CPC C12Q 1/6869; C12Q 1/6806; C12Q 1/6841; C12Q 1/6844

(57) ABSTRACT

The present invention provides methods to barcode nucleic acid for detection and sequencing. It applies a barcode template in a compartment with various targets, including nucleic acid fragments, nuclei and/or cells. After clonal amplification within the compartment, barcode sequence will integrate into its targets before the compartment is broken so that it will effectively barcode nucleic acid fragments originated from a nucleic acid fragment, a nucleus or a cell clonally. The barcode information can be used for tracking the origin of the fragment, nucleus or cell and be used for haplotype phasing and a variety of single cell-based applications including whole genome sequencing, targeted sequencing, RNA sequencing and immune repertoire sequencing.

### CROSS REFERENCE TO RELATED APPLICATIONS

This patent application is a U.S. National Phase patent application under 35 U.S.C. 371 of International Application No. PCT/US2021/018423 filed Feb. 17, 2021, which claims the benefit of and priority to U.S. Provisional Patent Application No. 62/977,618, filed on Feb. 17, 2020, both of which is incorporated herein by reference in their entirety. All publications, patents and other documents mentioned herein are incorporated by reference in their entirety.

### REFERENCE TO SEQUENCE LISTING

This specification includes a sequence listing submitted herewith, which includes the file entitled 168460-011901_ST25.txt having the following size: 2,964 bytes which was created Jan. 10, 2023, the contents of which are incorporated by reference herein.

### FIELD

The present invention relates in general methods for improved nucleic acid detection and sequencing for single cell analysis, haplotype phasing, de novo assembly and variant detection.

### BACKGROUND

The present invention is in the technical field of genomics. More particularly, the present invention is in the technical field of nucleic acid sequencing. Nucleic acid sequencing can provide information for a wide variety of biomedical applications, including diagnostics, prognostics, pharmacogenomics, and forensic biology. Sequencing may involve basic low throughput methods including Maxam-Gilbert sequencing (chemically modified nucleotide) and Sanger sequencing (chain-termination) methods, or high throughput next-generation methods including massively parallel pyrosequencing, sequencing by synthesis, sequencing by ligation, semiconductor sequencing, and others. For most sequencing methods, a sample, such as a nucleic acid target, needs to be processed prior to introduction into a sequencing instrument. For example, a sample may be fragmented, amplified or attached to an identifier. Unique identifiers are often used to identify the origin of a target. Most sequencing methods generate relatively short sequencing reads, ranging from tens of bases to hundreds of bases in length, and cannot generate complete haplotype phase information due to limited sequencing read length. Most biological samples contain many cells. And most assays are measuring responses for bulk cells, not at an individual cell level.

### SUMMARY

In one aspect, described herein are methods of tracking a target's origin by barcode tagging. The methods include encapsulating at least one unique barcode template with at least one target in a compartment; amplifying the barcode template(s) and modifying the target wherein modified target is capable of linking to a barcode in the compartment; linking a barcode sequence to a modified target so that a plurality of modified targets sharing the same one or more barcode sequences presented in the compartment; removing the compartments and collecting the barcode tagged modified targets for downstream applications. A target is selected from a group consisting of a nucleic acid, a protein including antibody, a ligand, a chemical compound, a nucleus, a cell, and a combination thereof. A cell can be prokaryotic or eukaryotic. The modification for a target is selected from a group consisting of strand transfer reaction, tagmentation reaction, reverse transcription, amplification, primer extension, restriction digestion, hybridization, ligation, fragmentation, and a combination thereof. In some embodiment, a target is treated and/or modified before encapsulation. A treatment is selected from a group consisting of denaturation, permeabilization, fixation, labeling, conjugation, in situ reactions, and a combination thereof. In some embodiment, compartment origin of different barcode sequences presented in the same compartment can be identified based on their shared compartment content.

In some embodiment, a barcode template comprises a central barcode sequence flanked by at least two handle sequences which can be used as priming site, hybridization site or binding site.

In one aspect, described herein are methods of tracking nucleic acid fragment origin by barcode tagging. The methods include providing a plurality of nucleic acid targets and a plurality of transpososomes, each transpososome comprises at least one transposon and one transposase; incubating nucleic acid targets and transpososomes together to form strand transfer complexes (STCs) on the nucleic acid targets; providing a plurality of unique barcode templates; compartmentalizing the nucleic acid targets with STCs and the barcode templates to generate two or more compartments comprising both one or more nucleic acid targets and one or more than one barcode templates with different barcode sequences; amplifying the barcode template in the compartment, fragmenting nucleic acid target by breaking the STCs to form tagmented nucleic acid fragments, and attaching barcode sequence to tagmented nucleic acid fragments so that a plurality of fragments sharing the same one or more barcode sequences presented in the compartment; removing the compartments and collecting the barcode tagged nucleic acid fragments.

In one aspect, described herein are methods of tracking nucleic acid fragment origin by barcode tagging. The methods include providing a plurality of nucleic acid targets and a plurality of transpososomes, each transpososome comprises at least one transposon and one transposase; incubating nucleic acid targets and transpososomes together to form strand transfer complexes (STCs) on the nucleic acid targets; providing a plurality of unique barcode templates; compartmentalizing the nucleic acid targets with STCs and the barcode templates to generate two or more compartments comprising both one or more nucleic acid targets and one or more than one barcode templates with different barcode sequences; attaching a barcode sequence to the nucleic acid targets in the compartment by i) fragmenting nucleic acid target by breaking the STCs to form tagmented nucleic acid fragments; ii) amplifying the tagmented nucleic acid fragments with non-target-specific primers (i.e. only transposon specific), and amplifying the barcode template(s); iii) linking a barcode template to a tagmented nucleic acid fragment, wherein a plurality of fragments sharing the same one or more barcode sequences presented in the compartment; removing the compartments and collecting the barcode tagged nucleic acid fragments for downstream applications. One of the applications is to generate haplotype phased sequencing information.

In one aspect, described herein are methods of tracking targeted nucleic acid fragment origin by barcode tagging. The methods include providing a plurality of nucleic acid targets, a plurality of target specific primers and a plurality of transpososomes, each transpososome comprises at least one transposon and one transposase; incubating nucleic acid targets and transpososomes together to form strand transfer complexes (STCs) on the nucleic acid targets; providing a plurality of unique barcode templates; compartmentalizing the nucleic acid targets with STCs and the barcode templates to generate two or more compartments comprising both one or more nucleic acid targets and one or more than one barcode templates with different barcode sequences; attaching a barcode sequence to the nucleic acid targets in the compartment by i) fragmenting nucleic acid target by breaking the STCs to form tagmented nucleic acid fragments; ii) amplifying the tagmented nucleic acid fragments with a transposon specific primer and a target-specific primer, and amplifying the barcode template(s); iii) linking a barcode template to a tagmented nucleic acid fragment, wherein a plurality of fragments sharing the same one or more barcode sequences presented in the compartment; removing the compartments and collecting the barcode tagged nucleic acid fragments. In some embodiments, the nucleic acid targets are within a cell or nucleus, wherein the cells or the nuclei are permeabilized or fixed, then incubated with a plurality of transpososomes before being compartmentalized with target specific primers and barcode templates.

In one aspect, described herein are methods of tracking targeted nucleic acid fragment origin by barcode tagging. The methods include providing a plurality of nucleic acid fragments, a plurality of unique barcode templates and a plurality of target specific primers wherein at least some said target specific primers are capable of attaching to barcode templates directly or indirectly; compartmentalizing the nucleic acid fragments, target specific primers and the barcode templates to generate two or more compartments comprising one or more nucleic acid fragments, target specific primers and one or more than one barcode templates with different barcode sequences; attaching a barcode sequence to the nucleic acid fragments in the compartment by i) amplifying the targets from the nucleic acid fragments using target-specific primers, and amplifying the barcode template(s); iii) linking a barcode template to an amplified nucleic acid target in the compartment, wherein a plurality of amplified nucleic acid targets sharing the same one or more barcode sequences presented in the compartment; removing the compartments and collecting the barcoded nucleic acid targets for further analyses including sequencing.

In one aspect, described herein are methods of single cell ATAC-seq. The methods include providing a plurality of cells or nuclei and a plurality of transpososomes, each transpososome comprises at least one transposon and one transposase; incubating them together to form strand transfer complexes (STCs) on accessible chromatin in the nuclei; providing a plurality of unique barcode templates; compartmentalizing the treated cells or nuclei and barcode templates to generate two or more compartments comprising both a cell or a nucleus and one or more than one barcode templates with different barcode sequences; amplifying the barcode template in the compartment, breaking cellular and/or nuclear membrane, fragmenting accessible chromatin by breaking the STCs to form tagmented nucleic acid fragments, and attaching barcode sequence to tagmented nucleic acid fragments so that a plurality of fragments sharing the same one or more barcode sequences presented in the compartment; removing the compartments and collecting the barcode tagged nucleic acid fragments; sequencing the barcode and barcode tagged nucleic acid to characterize the accessible chromatin region on a single cell basis.

In one aspect, described herein are methods of single cell ATAC-seq. The methods include providing a plurality of cells or nuclei and a plurality of transpososomes, each transpososome comprises at least one transposon and one transposase; incubating them together to form strand transfer complexes (STCs) on accessible chromatin in the nuclei; providing a plurality of unique barcode templates; compartmentalizing the treated cells or nuclei and barcode templates to generate two or more compartments comprising both a cell or a nucleus and one or more than one barcode templates with different barcode sequences; attaching a barcode sequence to accessible chromatin fragments in the compartment by i) breaking cellular and/or nuclear membrane, and fragmenting accessible chromatin by breaking the STCs to form tagmented nucleic acid fragments; ii) amplifying the said tagmented nucleic acid fragments and amplifying the barcode template; iii) linking a barcode template to a tagmented nucleic acid fragment, wherein a plurality of fragments sharing the same one or more barcode sequences presented in the compartment; removing the compartments and collecting the barcode tagged nucleic acid fragments; sequencing the barcode and barcode tagged nucleic acid to characterize the accessible chromatin region on a single cell basis.

In one aspect, described herein are methods of barcoding whole genome of a single cell. The methods include providing a plurality of cells or nuclei and fixing the cells or nuclei to dissociate DNA from the proteins inside the cells or nuclei; providing a plurality of transpososomes, each transpososome comprises at least one transposon and one transposase; incubating fixed cells or nuclei and the transpososomes together to form strand transfer complexes (STCs) on DNA inside the fixed cells or nuclei; providing a plurality of unique barcode templates; compartmentalizing the treated nuclei and barcode templates to generate two or more compartments comprising both a cell or a nucleus and one or more than one barcode templates with different barcode sequences; amplifying the barcode template in the compartment, breaking cellular and/or nuclear membrane, fragmenting the DNA by breaking the STCs to form tagmented nucleic acid fragments; attaching barcode sequence to tagmented nucleic acid fragments so that a plurality of fragments sharing the same one or more barcode sequences presented in the compartment; removing the compartments and collecting the barcode tagged nucleic acid fragments. In some embodiment, the strand transfer reaction happens after a cell or nucleus is compartmentalized with barcode template(s). The cells can be prokaryotic or eukaryotic.

In one aspect, described herein are methods of barcoding whole genome of a single cell. The methods include providing a plurality of cells or nuclei and fixing the cells or nuclei to dissociate DNA from the proteins inside the cells or nuclei; providing a plurality of transpososomes, each transpososome comprises at least one transposon and one transposase; incubating fixed cells or nuclei and the transpososomes to form strand transfer complexes (STCs) on DNA inside the fixed cells or nuclei; providing a plurality of unique barcode templates; compartmentalizing the treated nuclei and barcode templates to generate two or more compartments comprise both a cell or nucleus and one or more than one barcode templates with different barcode sequences; attaching a barcode sequence to said genomic DNA in said cells or nucleus in the compartment by i) breaking nuclear membrane, and fragmenting genomic DNA by breaking the STCs to form tagmented nucleic acid fragments; ii) amplifying the said tagmented nucleic acid fragments and amplifying the barcode template; iii) linking a barcode template to a tagmented nucleic acid fragment, wherein a plurality of fragments sharing the same one or more barcode sequences presented in the compartment; removing the compartments and collecting the barcode tagged nucleic acid fragments. In some embodiment, the strand transfer reaction happens after a cell or nucleus is compartmentalized with barcode template(s). The cells can be prokaryotic or eukaryotic.

In one aspect, described herein are methods for single cell targeted sequencing. The methods include providing a plurality of cells and/or nuclei, providing a plurality of unique barcode templates and providing a plurality of target specific primers, wherein at least some target specific primers are also capable of attaching to barcode templates directly or indirectly; compartmentalizing the cells and/or nuclei, the barcode templates and the target specific primers to generate two or more compartments comprising a cell and/or nucleus, one or more than one barcode templates with different barcode sequences, and target specific primers; amplifying the barcode template in the compartment, attaching the barcode sequence to target specific primers, breaking cell/nuclear membrane, priming target genomic regions with target specific primers to generate barcode attached target fragments so that a plurality of barcode attached target fragments sharing the same one or more barcode sequences presented in the compartment; removing the compartments and collecting the barcode attached target fragments; and sequencing the barcode and barcoded tagged nucleic acid to characterize the targeted regions per cell basis. Either DNA or RNA or both can be the target. When RNA is a target, reverse transcriptase will be included besides a DNA polymerase.

In one aspect, described herein are methods for single cell targeted sequencing. The methods include providing a plurality of cells and/or nuclei, providing a plurality of unique barcode templates, and providing a plurality of target specific primers, wherein said target specific primers is capable of attaching to barcode templates directly or indirectly; compartmentalizing the cells and/or nuclei, the barcode templates and the target specific primers to generate two or more compartments comprising a cell and/or nucleus, one or more than one barcode templates with different barcode sequences and target specific primers; attaching a barcode sequence to a targeted nucleic acid fragment in the compartment by i) breaking cell and/or nuclear membrane to release nucleic acids; ii) amplifying the nucleic acid targets and amplifying the barcode template; iii) linking a barcode template to an amplified nucleic acid target, wherein a plurality of nucleic acid targets sharing the same one or more barcode sequences presented in the compartment; removing the compartments and collecting the barcode attached target fragments; and sequencing the barcode and barcoded tagged nucleic acid to characterize the targeted regions per cell basis. Either DNA or RNA or both can be the target. When RNA is a target, reverse transcriptase will be included besides a DNA polymerase.

In one aspect, described herein are methods for single cell RNA sequencing. The methods include providing a plurality of cells or nuclei, providing a plurality of unique barcode templates, providing a reverse transcriptase and providing a plurality of primers, wherein the primers are capable as primers for cDNA synthesis, or for barcode template amplification, or for priming with cDNA, or for a combination thereof; unique molecular identifier (UMI) sequences can be incorporated in the primers for cDNA synthesis; compartmentalizing the cells, the barcode templates, the reverse transcriptase and the primers to generate two or more compartments comprise a cell, one or more than one barcode templates with different barcode sequences, reverse transcriptase and primers; in the compartment, lysing the cell, generating cDNAs, amplifying the barcode template, attaching said barcode sequence to cDNA fragments or fragments generated from cDNA so that a plurality of barcode attached fragments sharing the same one or more barcode sequences presented in the compartment; removing the compartments and collecting the barcode attached fragments; and sequencing the barcode and barcoded tagged nucleic acid to characterize cDNA profile on a single cell basis.

In one aspect, described herein are methods for single cell RNA sequencing. The methods include performing reverse transcription of RNA in situ; tagmenting cDNA in situ; compartmentalizing treated cells and barcode templates, each compartment comprises one treated cell and one or more than one barcode templates; amplifying barcode templates and tagmented cDNA, and coupling amplified barcode templates to tagmented cDNA in the compartment; removing the compartments and collecting the barcode attached fragments; sequencing the barcode and barcoded tagged nucleic acid to characterize RNA profile on a single cell basis. In some embodiment, nuclei instead of cells are used as the input material.

In one aspect, described herein are methods for single cell RNA sequencing. The methods include providing a plurality of cells, fixing and/or permeabilizing the cells; providing a reverse transcriptase and providing a plurality of primers, wherein the primers are capable as primers for cDNA synthesis; unique molecular identifier (UMI) sequences can be incorporated in the primers for cDNA synthesis; generating first strand and second strand cDNA in situ; providing a plurality of transpososomes, each transpososome comprises at least one transposon and one transposase, tagmentating double-stranded cDNA in situ; providing a plurality of unique barcode templates, compartmentalizing the treated cells, the barcode templates, and the primers to generate two or more compartments comprising a cell, one or more than one barcode templates with different barcode sequences, and primers; in the compartment, amplifying the barcode template and cDNA fragments, attaching a barcode sequence to a cDNA fragment or fragment generated from cDNA so that a plurality of barcode attached fragments sharing the same one or more barcode sequences presented in the compartment; removing the compartments and collecting the barcode attached fragments; and sequencing the barcode and barcoded tagged nucleic acid to characterize cDNA profile on a single cell basis. In some embodiment, nuclei instead of cells are used as the input material.

In one aspect, described herein are methods for single cell RNA sequencing. The methods include providing a plurality of cells, fixing and/or permeabilizing the cells; providing a reverse transcriptase and providing a plurality of primers, wherein the primers are capable as primers for cDNA synthesis; unique molecular identifier (UMI) sequences can be incorporated in the primers for cDNA synthesis; generating first strand cDNA in situ; providing a plurality of transpososomes, each transpososome comprises at least one transposon and one transposase, tagmenting RNA/cDNA hybrid in situ; compartmentalizing the cells, the barcode templates, and the primers to generate two or more compartments comprising a cell or nucleus, one or more than one barcode templates with different barcode sequences, and primers; in the compartment, amplifying the barcode template and tagmented cDNA fragments, attaching said barcode sequence to cDNA fragments or fragments generated from cDNA so that a plurality of barcode attached fragments sharing the same one or more barcode sequences presented in the compartment; removing the compartments and collecting the barcode attached fragments; and sequencing the barcode and barcoded tagged nucleic acid to characterize cDNA profile on a single cell basis. In some embodiment, nuclei instead of cells are used as the input material.

In one aspect, described herein are methods of analyzing both RNA and DNA in a single cell simultaneously. The methods include performing reverse transcription in situ for a plurality of cells, before or after cell fixation; performing strand transfer reaction in situ for these fixed cells; encapsulating these cells individually with one or more than one barcode templates in a compartment; amplifying the barcode templates, cDNA and DNA fragments in the compartment; coupling amplified barcode templates to cDNA and DNA fragments in the compartment; removing the compartments and collecting the barcode attached fragments; sequencing the barcode and barcoded tagged nucleic acid to characterize both RNA and DNA profile on a single cell basis. In some embodiment, nuclei instead of cells are used as the input material.

In one aspect, described herein are methods of analyzing gene expression and gene regulation in a single cell simultaneously or RNA-seq and ATAC-seq in a single cell simultaneously. The methods include performing reverse transcription in situ for a plurality of cells; performing strand transfer reaction in situ for these cells; encapsulating these cells individually with one or more than one barcode templates in a compartment; in some embodiment, the cells are fixed before encapsulation; amplifying the barcode templates, cDNA and accessible chromatin DNA fragments in the compartment; coupling amplified barcode templates to cDNA and chromatin DNA fragments in the compartment; removing the compartments and collecting the barcode attached fragments; sequencing the barcode and barcoded tagged nucleic acid to characterize both RNA and accessible chromatin DNA profile on a single cell basis. In some embodiments, in situ strand transfer reaction is performed before reverse transcription reaction.

In one aspect, describe herein are methods of CITE-seq using encapsulated barcode amplification and barcode tagging transcripts and nucleic acid labeled epitopes.

In one aspect, described herein are methods of identifying the compartment origin of any barcodes when there are more than one barcodes in a compartment when partitioning barcode templates and barcoding targets. Providing a compartment content specific information, identifying both barcode information of a target and compartment content information of the barcode, and grouping the barcodes with the same compartment content information to collect all the targets associated with these barcodes.

In one aspect, the compartment content information is shared breakpoint coordinates of tagmented fragments from more than one nucleic acid fragments, or shared UMI sequence from more than one target, or combination thereof.

### BRIEF DESCRIPTION OF THE DRAWINGS

FIG. 1 illustrates a nucleic acid barcoding method using transpososomes and barcode templates with compartmentation reaction. BC means a barcode on a barcode template.

FIG. 2 illustrates methods to attach clonally amplified barcode template to tagmented nucleic acid fragment in a compartment. A. Amplified barcode templates are used as primers to further amplify a target (200) in order to attach the barcode to the target in the compartment. B. A linker oligo (203) is used to couple amplified barcodes to a target (200) indirectly so that after amplification a barcode sequence is attached to the target. C. Dual amplification of a barcode template and a target (200) in a compartment separately (204, 205) and couple an amplified barcode sequence to an amplified target (206, 207). D. Dual amplification of two barcode templates and a target (200) in a compartment separately (210, 213) and couple an amplified barcode sequence to an amplified target (214,215). BC means a barcode on a barcode template. BC1 and BC2 are different barcode sequences.

FIG. 3 illustrates a single cell ATAC-seq library preparation method using transpososomes tagged nuclei and barcode templates with compartmentation reaction.

FIG. 4 illustrates a single cell whole genome barcoding method using transpososomes tagged fixed nuclei and barcode templates with compartmentation reaction.

FIG. 5 illustrates a method to enrich targeted regions using barcoded nucleic acid fragments and target specific primer set.

FIG. 6 illustrates that barcoded single cell can significantly improve detection power of somatic mutation with the combined ability for individual cell identification and sequencing error correction with unique molecule identification (UMI).

FIG. 7 illustrates a single cell RNA-seq method with both in situ reactions and compartmentalized barcode amplification and coupling reaction.

FIG. 8 illustrates a single cell nucleic acid barcoding reaction for targeted sequencing in a compartment.

FIG. 9 shows sequencing library preparation workflow for same cell ATAC-seq and 3′ RNA-seq analysis.

FIG. 10 illustrates clonal barcoding reactions in a droplet through dual amplification of barcode template(s) and tagmented fragments and attaching amplified barcode templates to tagmented fragments.

FIG. 11 illustrates linked read sequencing results. A. Sequencing read count histogram of same barcode Read 1 read distance to the next Read 1 alignment to demonstrate a linked-read feature from a whole genome linked read sequencing of an E. coli sample. B. Sequencing coverage of each genomic DNA molecule by linked reads from a linked read sequencing of a pool of 4 kb HLA amplicons.

FIG. 12 shows a TapeStation high sensitivity D1000 screen tape profile of a cleaned up single cell ATAC-seq library.

FIG. 13 shows some Cell Ranger analysis results of a single cell ATAC-seq experiment.

Transposases in the figures are showed as a tetramer or dimer which is for illustration only. Different transposases can be used in the reaction.

### DETAILED DESCRIPTION

Most commercially available sequencing technologies have limited sequencing read length. Second generation high throughput sequencing technologies can sequence only several hundred bases and rarely reach a thousand bases. However, nucleic acid sequences of a gene can span from several kilobases to tens and hundreds of kilobases, which means sequencing read length of tens of kilobases is necessary to successfully determine the haplotypes of all genes.

Meanwhile, most sequencing today are bulk sequencing of DNA or RNA extracted from many cells at once although individual cells are different. By using averaged molecular or phenotypic measurements of a cell population to represent an individual cell behavior, conclusions could be biased by the expression profiles of a majority group of cells or over-expressed outliers; and we will not have the sensitivity to identify all unique patterns from an individual cell which could be distinctive functional behaviors for a cell at a given location and time. In addition, early tumor detection has been significantly restrained by limited ability to detect very low frequent somatic mutation currently due to presence of high background wild type signal from normal cells or tissue. However, with improved ability to identify every single cell, we will be able to separate the mutant tumor cells from wild type cells by genotyping at single cell level. This will remove the wild type background signal generated from normal cells almost completely and make somatic mutation detection as easy as germline mutation detection.

Both Tn5 transpososome and MuA transpososome have been previously described to simultaneously fragment DNA and introduce adaptors at high frequency in vitro, creating sequencing libraries for next-generation DNA sequencing (Adey et al 2010, Caruccio et al 2011, and Kavanagh et al 2013). These specific protocols remove any phasing or contiguity information as a result of the fragmentation of the DNA. In these protocols after DNA reaction with transpososomes, a column purification, a heat treatment step, a protease treatment or an incubation with SDS solution or EDTA solution was necessary to release the transposase from the strand transfer complexes (STC) so that DNA is tagmented into fragments. It has been known that MuA transpososome can form a very stable STC when attack DNA targets (Surette et al 1987, Mizuuchi et al 1992, Savilahti et al 1995, Burton and Baker 2003, Au et al 2004). Similar stability has also been observed for Tn5 transpososome during transposition reaction (Amini et al 2014).

This invention takes advantage of the stability of STC and clonal barcode generation by compartmentation amplification and provides methods to uniquely barcode nucleic acid targets sub-fragments and/or barcode nucleic acid in a single cell.

The term “adaptor” as used herein refers to a nucleic acid sequence that can comprise a primer binding sequence, a barcode, a linker sequence, a sequence complementary to a linker sequence, a capture sequence, a sequence complementary to a capture sequence, a restriction site, an affinity moiety, unique molecular identifier, and a combination thereof.

The term “amplification” as used here refers to a process to generate multiple copies of an original template. The method for amplification is selected from the group consisting of PCR, RPA, MALBAC, and isothermal amplification methods for both linear amplification and exponential amplification.

A “barcode template”, which contains a barcode sequence, flanked by at least one handle sequence at one end or two handle sequences at both ends. Length of barcode sequence ranges from 4 bases to 100 bases. The handle sequences can be used as binding sites for hybridization or annealing, as priming sites during amplification, or as binding site for sequencing primers or transposase enzyme. Furthermore, barcode sequences can be selected from a pool of known nucleotide sequences or randomly chosen from randomly synthesized nucleotide sequences. A barcode template can be a DNA, an RNA or a DNA/RNA hybrid.

The term “transposase” as used herein refers to a protein that is a component of a functional nucleic acid protein complex capable of transposition and which is mediating transposition, including but not limited to Tn, Mu, Ty, and Tc transposases. The term “transposase” also refers to integrases from retrotransposons or of retroviral origin. It also refers to wild type protein, mutant protein and fusion protein with tag, such as, GST tag, His-tag, etc. and a combination thereof.

The term “transposon”, as used herein, refers to a nucleic acid segment that is recognized by a transposase or an integrase and is an essential component of a functional nucleic acid-protein complex capable of transposition. Together with transposase they form a transpososome and perform a transposition reaction. It refers to both wild type and mutant transposon.

A “transposable DNA” as used herein refers to a nucleic acid segment that contains at least one transposon unit. It can also comprise an affinity moiety, un-natural nucleotides and other modifications. The sequences besides the transposon sequence in the transposable DNA can contain adaptor sequences.

The term “transpososome” as used herein refers to a stable nucleic acid and protein complex formed by a transposase non-covalently bound to a transposon. It can comprise multimeric units of the same or different monomeric unit.

A “transposon joining strand” as used herein means the strand of a double stranded transposon DNA that is joined by the transposase to the target nucleic acid at the insertion site.

A “transposon complementary strand” as used herein means the complementary strand of the transposon joining strand in the double stranded transposon DNA.

A “strand transfer complex (STC)” as used herein refers to a nucleic acid-protein complex of transpososome and its target nucleic acid into which transposons insert, wherein the 3′ ends of transposon joining strand are covalently connected to its target nucleic acid. It is a very stable form of nucleic acid and protein complex and resists heat and high salt in vitro (Burton and Baker, 2003).

A “strand transfer reaction” as used herein refers to a reaction between a nucleic acid and a transpososome, in which strand transfer complexes form.

A “tagmentation reaction” as used herein refers to fragmentation reaction where transpososomes insert into a target nucleic acid through strand transfer reaction and form strand transfer complexes, and strand transfer complexes are then broken under certain conditions, such as, protease treatment, high temperature treatment, or a protein denaturing agent, e.g. SDS solution, guanidine hydrochloride, urea, etc., or a combination thereof, so that the target nucleic acid breaks into small fragments with transposon end attached.

A “reaction vessel” as used herein means a substance with a contiguous open space to hold liquid; it is selected from the group consisting of a tube, a well, a plate, a well in a multi-well plate, a slide, a spot on a slide, a droplet, a tubing, a channel, a bottle, a chamber and a flow-cell.

Encapsulating Nucleic Acid with Strand Transfer Complexes and Barcode Templates in Water-In-Oil Emulsion Droplets

This invention provides a method to encapsulate nucleic acid targets with STCs and a barcode template in water-in-oil emulsion droplets, and further generate barcode tagged nucleic acid fragments.

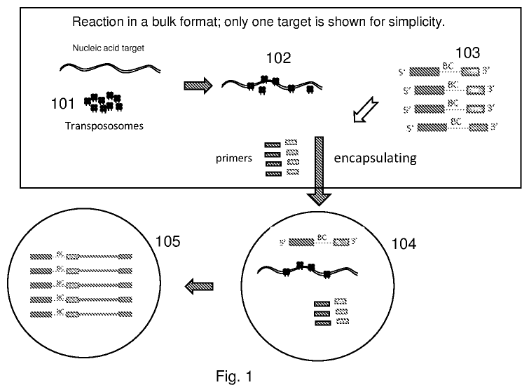

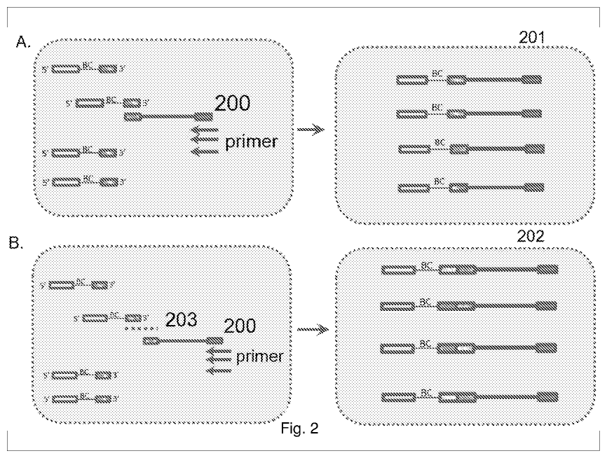

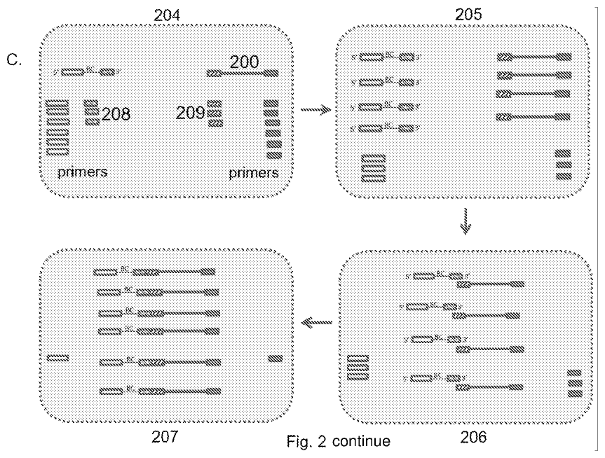

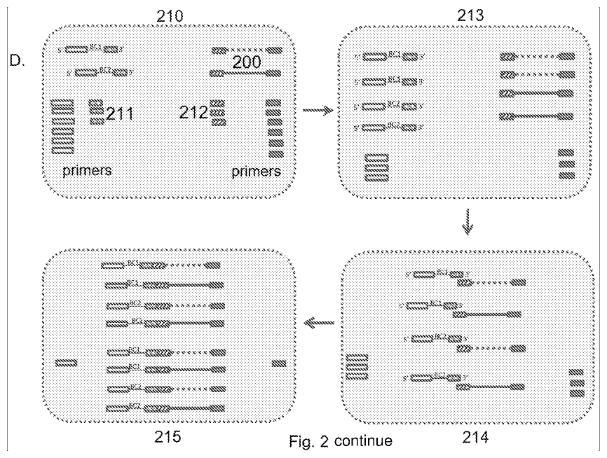

Nucleic acid targets are reacted with transpososomes (101) and form stable strand transfer complexes (102) while keep the contiguity of nucleic acid targets (FIG. 1). The nucleic acid targets are double-stranded. In some embodiment, they are double stranded DNA. In some embodiments, they are DNA and RNA hybrid. The strand transfer reactions happen with a plurality of nucleic acid targets in one reaction vessel. In some embodiment, one type of transpososome is used; in other embodiments, more than one types of transpososome are used simultaneously or sequentially. The nucleic acid targets with STCs (102) are mixed with a plurality of barcode templates (103) in the solution. In some embodiment, each barcode template has a unique barcode sequence and different from others. In some embodiment, for most barcode templates, each has a unique barcode sequence and different from others. At least one of the transposable DNA in the transpososome is capable of hybridizing to one end of barcode template directly (FIG. 2A) or indirectly with a linker and/or a primer (FIG. 2B). Additional enzymes and substrates, such as, DNA polymerase, dNTP and primers are also provided in an aqueous solution in the same reaction vessel. In some embodiment, primers are used to amplify the barcode template. In some embodiment, primers can be used to amplify tagmented nucleic acid target fragments. Amplification includes exponential amplification and linear amplification. In some embodiment, different primers can be used to amplify the barcode template and tagmented nucleic acid target fragments in parallel (FIG. 2C), then the two groups of amplified products are capable to merge/couple into one piece via shared homology between the two inner primers (FIG. 2C, 208 and 209) or via an additional linker which is capable to bridge a barcode template and a tagmented fragment together. Water-in-oil emulsion droplets (104) are generated in such conditions that one to a few nucleic acid targets with STCs are mixed with one barcode template in one droplet. Proper titration of nucleic acid targets with STCs and barcode templates can be used here based on the Poisson distribution. In some embodiment, more than one barcode templates with different barcode sequences can be used in an emulsion droplet and it will significantly increase the barcode presence in the emulsion droplets and number of droplets with positive products so that increase the reaction yield significantly. In some embodiment, when both barcode templates and tagmented fragments are amplified before attaching a barcode sequence to a tagmented fragment, more than one barcode templates with different barcode sequences in the same emulsion droplet will not affect the true representation of the nucleic acid targets at all if different barcodes are randomly attached to the amplified copies of tagmented fragments (FIG. 2D). In this way, most emulsion droplets will contain barcode template, which will be available for barcode attachment to nucleic acid target when the target is also present in the same droplet. This makes it feasible to get almost 100% droplets which contain any nucleic acid target be useful for reaction. The emulsion droplets have a diameter from 1 μm to 200 μm, and preferably from 5 μm to 30 μm. When more than one barcodes are in an emulsion droplet compartment, these barcodes can be traced to one original compartment by utilizing the breakpoint coordinates of the tagmented fragments. Specifically, the breakpoints created by transposase tagmentation are different among different nucleic acid targets. If DNA fragments attached with a barcode share the same breakpoint coordinates with fragments attached with one or more other barcodes, these fragments are likely from the same original compartment. For a plurality of nucleic acid targets in an experiment, there is a possibility that two different nucleic acid fragments will produce the same breakpoint after transposase tagmentation. The chances for such collision are much lower when multiple breakpoints are used for discrimination. In some embodiments, UMI labeled transpososome can be used during strand transfer reaction or tagmentation reaction to increase the uniqueness of the fragment for identification. The UMI information can be used for compartment identity when different barcodes share many fragments with the same set of UMI population beside the same set of fragment breakpoints.

After a heat treatment, such as, at 60° C. to 75° C. for about 5-10 minutes, transposase will be released from the STCs and nucleic acid target breaks into smaller fragments. When still in a water-in-oil droplet, a DNA polymerase will fill in the gaps left during the transposition reaction. Emulsion amplification is performed to amplify barcode template in the droplet. Amplified barcode templates will hybridize to the tagmented fragments directly (FIG. 2A) or indirectly (FIG. 2B) and attach the barcode sequence to the fragments (105, 201, and 202) during amplification reaction. In some embodiment, unique molecular identifiers (UMIs) are added to the barcode templates during emulsion reaction. In some embodiment, UMIs are integrated as a linker (203) or a primer (209 and 212) in FIG. 2. After emulsion amplification reaction, emulsion droplets are broken by high salt, detergent, alcohol, organic chemicals or combination of these. Aqueous phase solution is collected. In some embodiment, one or more biotinylated primers are used so that amplified barcoded fragments can be pulled out easily with streptavidin beads. In some embodiment, one or more biotinylated dNTPs are used in the emulsion amplification. In some embodiment, primers with sample-specific barcode are used in the emulsion droplets during emulsion amplification so that emulsion amplification products from different sample reactions can be pooled together for final amplification or adaptor modification to make sequencing ready libraries.

In some embodiment, the nucleic acid targets are whole genomic DNA. This barcoding method can be used for de novo sequencing, whole genome haplotype phasing and structural variant detection. In some embodiments, the nucleic acid targets are DNA fragments, cDNA or a portion of captured DNA by hybridization capture, primer extension or PCR amplification. This barcoding method will be able to phase the variants of these DNA molecules. In some embodiment, target specific primers can be used in the compartment to amplify specific nucleic acid targets with or without reaction with transpososomes.

### Encapsulating Transposase Tagged Cells or Nuclei and Barcode Template in Water-In-Oil Emulsion Droplets

This invention provides a method to encapsulate cells or nuclei after strand transfer reaction and a barcode template in water-in-oil emulsion droplets, and further generate barcode tagged nucleic acid fragments for single cell level analysis.

ATAC-seq (Assay for Transposase-Accessible Chromatin using sequencing) is gaining more and more popularity as a state-of-the-art molecular biology tool to assess genome-wide chromatin accessibility (Buenrostro et al, 2013). ATAC-seq identifies accessible chromatin regions by tagging open chromatin with a hyperactive mutant Tn5 transposase that integrates sequencing adaptors into open regions of the genome. The tagged DNA fragments are purified, amplified by PCR and sequenced. Sequencing reads are then used to infer regions of increased accessibility as well as to map regions of transcription-factor binding sites and nucleosome positions. While natural wild type transposases have a low level of activity, ATAC-seq employs a mutated hyperactive transposase (Reznikoff et al, 2008), which has been successfully adapted to efficiently identify open chromatin and identify regulatory elements across the genome. Furthermore, single cell ATAC-seq is to separate single nuclei and perform ATAC-seq reactions individually (Buenrostro et al, 2015). Higher throughput single cell ATAC-seq uses combinatorial cellular indexing to measure chromatin accessibility in thousands of individual cells. Single-cell ATAC seq enables the identification of cell types and states for developmental lineage tracing. ATAC-seq will likely be a key component of comprehensive epigenomic workflows.

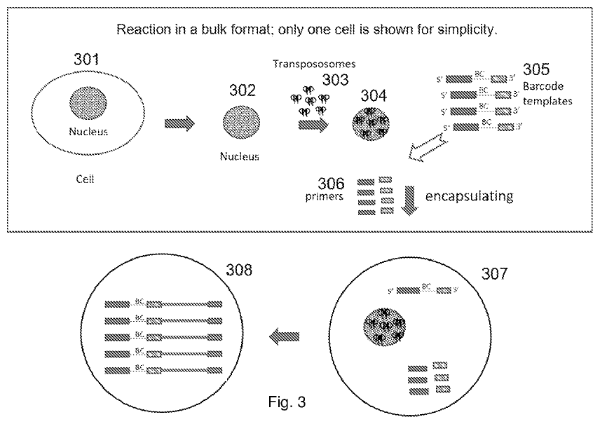

This invention uses emulsion method to encapsulate a transposase treated nucleus and a unique barcode template, then clonally amplify the barcode template within an emulsion droplet and attach the clonally amplified barcodes to tagmented accessible DNA fragments (FIG. 3). The tagmented DNA can also be amplified in the emulsion droplet. This barcoding method offers a high throughput and low-cost cellular indexing for single cell ATAC-seq analysis.

In some embodiment, nuclei (302) are collected from cells or tissue samples and incubated with transpososomes to form STCs (304), then mixed with a plurality of different barcode templates in a bulk reaction (FIG. 3). In some embodiment, whole cells are treated with transpososomes to form STCs inside the nuclei without isolation of nuclei. In some embodiment, the transpososome comprises a mutated hyperactive TN5 transposase. In some embodiment, the transpososome comprises a MuA transposase. Other enzymes and substrates, such as, DNA polymerase, dNTP and primers are also provided in an aqueous solution in the same bulk reaction. Water-in-oil emulsion droplets are generated in such conditions that one nucleus and one barcode template are present in most droplets by limiting titration or partitions based on Poisson distribution (307). The emulsion droplets have a diameter from 10 μm to 200 μm, and preferably from 20 μm to 60 μm. After a heat treatment, such as, at 60° C. to 75° C. for about 5-10 minutes, transposase will be released from the STCs and nucleic acid targets break into smaller tagged fragments. When still in a water-in-oil droplet, a DNA polymerase will fill in the gaps left during the transposition reaction on the tagged fragments. Nuclear membrane will break during emulsion PCR denaturing step, and emulsion amplification is performed to amplify barcode template in the droplet. Amplified barcode templates are capable to hybridize to the tagmented fragments directly or indirectly and attach the barcode sequence to the fragments during amplification reaction. In some embodiment, both barcoded templates and tagmented fragments are amplified parallelly first, then merged or coupled together to form barcoded tagmented fragments as FIGS. 2C and 2D. After emulsion amplification reaction, emulsion droplets are broken by high salt, detergent, alcohol, organic solution or combination of these. Aqueous phase solution is collected. In some embodiment, one or more biotinylated primers or one or more biotinylated dNTPs are used so that amplified barcoded fragments can be pulled out easily with streptavidin beads. Sequencing library prepared from these barcoded fragments will be a single cell ATAC-seq library.

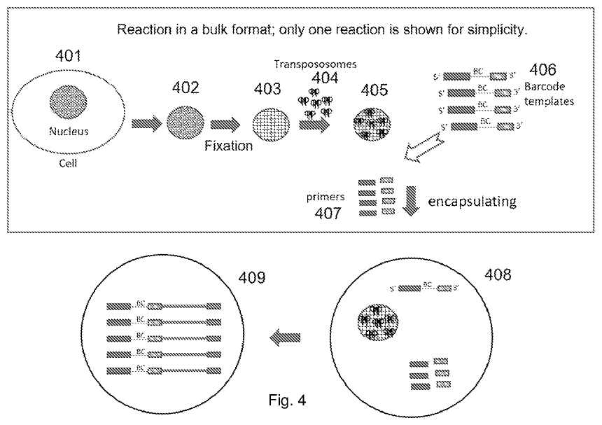

Besides single cell ATAC-seq application, this invention also provides a single cell whole genome sequencing method after modifications. It uses emulsion method to encapsulate an alcohol fixed nucleus treated with transposase and a unique barcode template, and clonally amplify the barcode template within an emulsion droplet and attach the barcodes to tagmented genomic DNA fragments (FIG. 4).

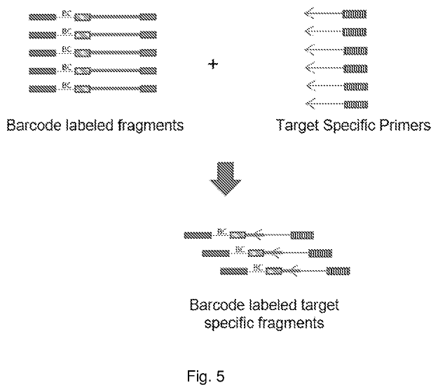

In some embodiment, nuclei (402) are collected from cells or tissue samples and fixed with alcohol-based fixation. Alcohol based fixative or Hepes-glutamic acid buffer-mediated organic solvent protection effect (HOPE) fixative or other similar fixative will be able to denature the proteins in the nuclei but keep the nucleic acid intact. In this way, it will be able to expose all the genomic DNA from the chromatin. In some embodiment, fixed cells are used directly without isolation of nuclei. After washing away fixation solution, nuclei are treated with transpososomes to form STCs (405) on the genomic DNA, then mixed with a plurality of different barcode templates in a bulk reaction. Other enzymes and substrates, such as, DNA polymerase, dNTP and primers are also provided in an aqueous solution in the same bulk reaction. Water-in-oil emulsion droplets are generated in such conditions that one nucleus and one barcode template are present in a droplet by limiting titration or partitions based on Poisson distribution (408). The emulsion droplets have a diameter from 10 μm to 200 μm, and preferably from 20 μm to 60 μm. After a heat treatment, such as, at 60° C. to 75° C. for about 5-10 minutes, transposase will be released from the STCs and nucleic acid target breaks into smaller tagmented fragments. When still in a water-in-oil droplet, a DNA polymerase will fill in the gaps left during the transposition reaction. Nuclear membrane will break during emulsion amplification. Emulsion amplification is performed to amplify barcode template in the droplet. Amplified barcode templates are capable to hybridize to the tagmented fragments directly or indirectly and attach the barcode sequence to the fragments during amplification reaction. In some embodiment, both barcoded templates and tagmented fragments are amplified parallelly first, then merged together to form barcoded tagmented fragments as FIGS. 2C and 2D. After emulsion amplification reaction, emulsion droplets are broken by high salt, detergent, alcohol, organic reagents or combination of these. Aqueous phase solution is collected. In some embodiment, one or more biotinylated primers or one or more biotinylated dNTPs are used so that amplified barcoded fragments can be pulled out easily with streptavidin beads. In some embodiment, library prepared from these barcoded fragments can be used directly for single cell whole genome sequencing and single cell CNV analysis. In some embodiment, library prepared from these barcoded fragments can be used for further targeted capture of whole exome or smaller targeted region for targeted sequencing (FIG. 5). In some embodiment, cells from a metagenomic sample are used in this barcoding reaction directly. Prokaryotic cell wall can be permeabilized enzymatically and/or chemically. This single cell sequencing method eliminates the need of genomic DNA preparation which is a bottleneck for metagenomic sample preparation and keep high molecular weight DNA intact in the cells directly so that it can improve the assembly efficiency. This method will preserve the organism composition in a metagenomic sample very well and improve the accuracy of the measurement of organism composition using cell level information based on barcode instead of only genomic DNA level information which contains more bias due to accessibility, amplification, or sequencing.

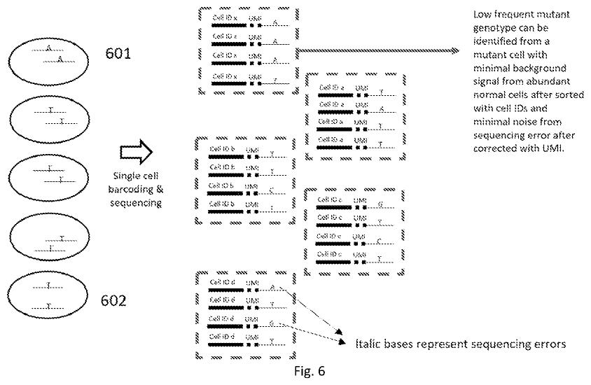

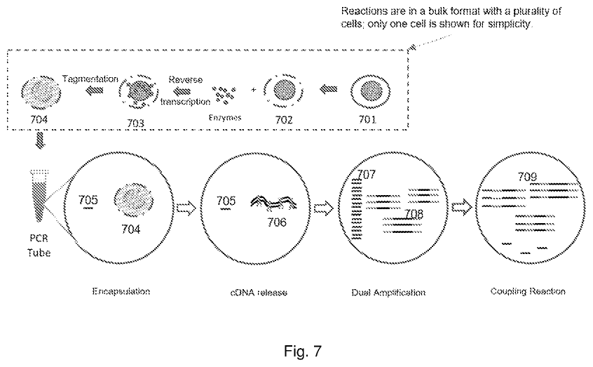

One advantage of this kind of single cell targeted sequencing is that it has much higher sensitivity for low frequent variant detection, such as, somatic mutation detection (FIG. 6). With the ability to uniquely barcoding individual cells, we can detect any mutations at a single cell level, which will effectively eliminate the background noise from surrounding cells. This enables very high sensitivity for detecting very low frequent somatic mutations which is required for early cancer detection. FIG. 6 illustrates the power of genotyping at a single cell level. There is a cell containing a mutant allele A (601), but there are many wild type cells containing a normal allele T (602) in the same sample. Unique molecular identifiers (UMIs) are added in the barcoding reactions. With the incorporation of molecule specific UMI during single cell barcoding and sequencing, sequencing reads can be grouped based on their cell ID first, and for each cell, we are able to identify sequencing error based on UMI and make a correct variant call easily. This approach can be applied for circulating tumor cells, tissue biopsy samples or tissue sections.

In some embodiment, more than one barcode templates with different barcode sequences can be presented in an emulsion droplet to increase the capture rate. When more than one barcodes are present in the emulsion droplet and shared by one nucleus or cell, these barcodes can be traced back to their original nucleus or cell by utilizing the breakpoint coordinates of the tagmented fragments. Specifically, the breakpoints created by transposase tagmentation are different among different nuclei or cell. If DNA fragments attached with a barcode share the same breakpoint coordinates with fragments attached with one or more other barcodes, these barcodes are likely from the same original nucleus or cell. There is a possibility that two nuclei or cells will produce the same breakpoint in some fragments after transposase tagmentation. The chances for such collision are much lower when multiple breakpoints are used for discrimination. The more shared breakpoint coordinates among two barcodes, the higher confidence that these two barcodes are from the same compartment, i.e. the same cell or nucleus. In some embodiments, the randomness of the tagmentation breaking point is used as a UMI function to track duplication arisen from the amplification and improve the counting accuracy of unique target.

Beside the single cell genomic DNA analysis described above, this invention can also be used for single cell RNA analysis. In some embodiment, a reverse transcriptase and cDNA primers as the first set of primers can be included in the emulsion reaction. In some embodiment, cDNA primers have poly T sequence at the 3′ end; in some embodiment, cDNA primers have GGG at the 3′ end; in some embodiment, cDNA primers have target specific primers at the 3′ end. In some embodiment, cDNAs are synthesized using mRNA as templates; in some embodiment, cDNAs are synthesized using other RNA species as templates. During the early phase of emulsion reaction, cDNA or partial cDNA will be generated from mRNA in the single cell or nucleus by reverse transcriptase. The barcoding reaction will proceed as described previously except using the cDNA as input DNA. With different primers used for reverse transcription or cDNA priming, this method can be modified for single cell transcriptome analysis, single cell 3′ RNA-Seq analysis, single cell 5′ RNA-Seq analysis, single cell target-seq application, and immune repertoire analysis.

When combining in situ reactions for bulk cells and encapsulation of individual treated cell with one or more barcode template for compartmentalized amplification and barcode tagging reaction, this invention provides another high throughput method for single cell RNA analysis. Cells (701) are permeabilized (702). In some embodiment, RNAs in the permeabilized cells (702) are transcribed to cDNAs by reverse transcriptase in situ (703). A second strand DNA is synthesized to form a double-stranded DNA as input for tagmentation in situ. In some embodiment, RNAs in the cells are transcribed to first strand cDNAs by reverse transcriptase in situ. RNA/cDNA hybrid double strand is used as input for tagmentation in situ (704). In some embodiment, cDNA primers have poly T sequence at the 3′ end; in some embodiment, cDNA primers have GGG at the 3′ end; in some embodiment, cDNA primers have target specific primers at the 3′ end; in some embodiment, cDNAs are synthesized using mRNA as templates; in some embodiment, cDNAs are synthesized using other RNA species as templates. The treated cells containing in situ tagmented cDNA (704) will be encapsulated with one or more barcode templates (705) for clonal amplification reaction. During the clonal reaction, tagmented cDNA fragments (706) will be released from the cells, both barcode template(s) and tagmented cDNA are amplified (dual amplification) and amplified barcode templates (707) are coupling to the amplified cDNA fragments (708) and a plurality of barcode attached fragments sharing the same one or more barcode sequences presented in the compartment are generated (709). With different primers used for reverse transcription or cDNA priming, this method can be modified for single cell transcriptome analysis, single cell 3′ RNA-Seq analysis, single cell 5′ RNA-Seq analysis, single cell target-seq application, and immune repertoire analysis.

In some embodiment, more than one barcode templates with different barcode sequences can be presented in an emulsion droplet to increase the cell capture rate. When more than one barcode templates are presented in an emulsion droplet and shared by one cell or nucleus in the compartment, these barcodes can be traced to one original cell/nucleus by the UMI on the reverse transcription primer.

### Encapsulating Cells, Barcode Templates and Target-Specific-Primers in Water-In-Oil Emulsion Droplets

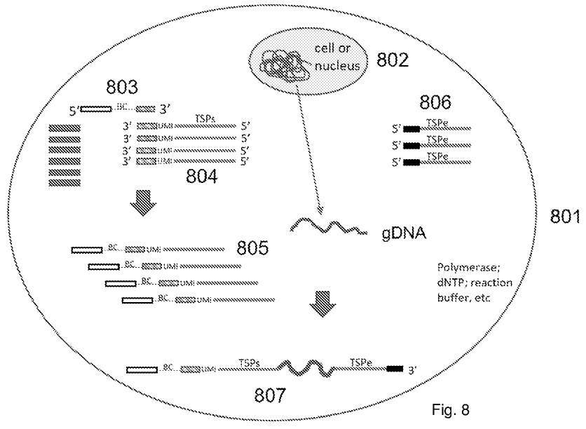

This invention provides a high throughput method for single cell targeted sequencing. Isolated cells or nuclei (802) are encapsulated with unique barcode templates (803) and first set of target specific primers (804) by emulsion droplets (FIG. 8). Additional enzymes and substrates, such as, DNA polymerase, dNTP and common primers are also provided in the aqueous solution. Water-in-oil emulsion droplets (801) are generated in such conditions that one cell or one nucleus and one barcode template are present in a droplet by limiting titration or partitions based on Poisson distribution. The emulsion droplets have a diameter from 10 μm to 200 μm, and preferably from 20 μm to 100 μm. Cell membrane or nuclear membrane will break during emulsion amplification and release genomic DNA into emulsion droplets. Emulsion amplification is performed to amplify barcode template and attach target specific primers to barcode template in the droplet. Single stranded amplified barcode templates with target specific sequences at 3′ end (805) are capable to hybridize to the genomic DNA targets and make copies of targeted region during amplification reaction. In some embodiment, a second set of target specific primers (806) are included in the aqueous solution during emulsion droplet generation. After emulsion amplification reaction, barcode tagged amplicons of the targets (807) will be generated, which can be used for sequencing library preparation and sequencing analysis. In some embodiment, to reduce primer dimers generated during amplification, dUTP containing primers can be used and in combination with UDG/APE1/Exol treatment after emulsion amplification. Sequencing library adaptor can be added by ligation after cleaning up primer dimers.

### Method for Analyzing RNA and DNA in the Same Cell

Currently most single cell methods are only capable for separated RNA or DNA analysis for different single cell. In another word, they can't analyze both RNA and DNA from the same cell at the same time.

The invention described here can be easily used to monitor RNA expression and determine DNA genotype for the same cell simultaneously. In some embodiment, cells after in situ reverse transcription reaction to generate cDNA are fixed to dissociate DNA from protein. In some embodiment, cells are fixed first before in situ reverse transcription happens. Poly T primers can be used to capture 3′ mRNA. In some embodiment, a UMI sequence is associated with the poly T primers. Strand transfer reaction or tagmentation reaction can be performed in situ inside the treated cells or after the cells are encapsulated with barcode templates in a compartment. In some embodiment, strand transfer reaction or tagmentation reaction is not necessary if targets are all specific. During the cell encapsulation, cDNA specific primer and DNA target specific primers and/or transposon specific primers are enclosed with primers for amplifying barcode templates at the same time. In some embodiment, cDNA amplification is for 3′ mRNA when using poly T primers. DNA amplification is target specific or for whole genome. After amplification of barcode template(s), cDNA and DNA fragments, barcode templates are linked to amplified cDNA or DNA fragment in the compartment. Barcode tagged cDNA and DNA will be released from the compartment and collected for further analysis on gene expression and genomic variation.

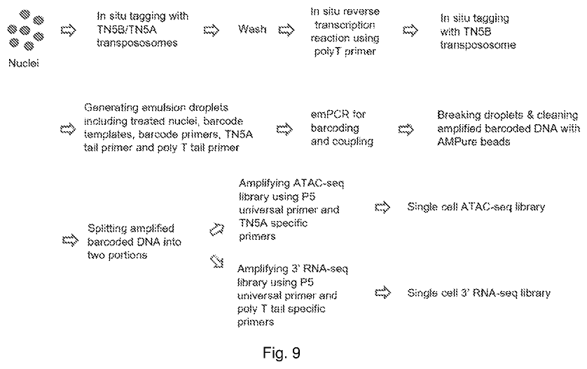

This invention also provides a method for simultaneous ATAC-seq and RNA-seq of the same cell. Cells are permeabilized and reverse transcription using poly T primer to generate cDNA are performed in situ. In some embodiment, the cDNAs are first strand cDNA only. In some embodiment, the cDNAs are after second strand cDNA synthesis. Incubate these cells with transpososomes for strand transfer reaction at open chromatin sites inside nuclei and with cDNA in the cells. In some embodiments, strand transfer reaction at open chromatin sites is performed before reverse transcription. Encapsulate these cells individually with one or more barcode templates in a compartment for barcode amplification and tagmented RNA and DNA amplification. In some embodiment, these cells are fixed to denature cellular proteins and exogenous reverse transcriptase and transposase before encapsulation. In some embodiments, nuclei are isolated from cells before strand transfer reaction and/or reverse transcription reaction (FIG. 9).

Cellular Indexing of Transcriptomes and Epitopes by Sequencing (CITE-seq) is a multimodal single cell phenotyping method, which uses DNA-barcoded antibodies to convert detection of proteins into a quantitative, sequencable readout. Antibody-bound oligos act as synthetic transcripts that are captured during most large-scale oligo dT-based single cell RNA-seq library preparation protocols (Stoeckius et al, 2017). For our method above, when cDNA primer is ployT type design, CITE-seq type library will be able to be generated efficiently.

In some embodiments, instead of a nucleic acid, a genome, a protein, a nucleus, a cell or a microbe, encapsulated target is a protein complex, a protein and nucleic acid complex, a small molecule, a macromolecule, a chemical compound, a ligand, a particle, a microparticle, or a combination thereof, wherein they are labeled or attached with a nucleic acid as their identification or marker.

Although the compartmentation method described in this invention is encapsulation in a water-in-oil emulsion, other sequestering methods are also feasible. Certain type of liposomes, such as, giant unilamellar liposome vesicles (GUVs) with a size from 1-200 um in diameter, have showed very high thermostable and are able to perform PCR amplification inside of its enclosure (Kurihara et al 2011, Laouini et al 2012). In some embodiments, the emulsion droplets used for compartment generation in this invention can be replaced by GUVs. In some embodiments, compartmentation is achieved by microwells. In some embodiments, compartmentation is achieved by openarray. In some embodiments, compartmentation is achieved by microarray, microtiter plate or other physically separated compartmentation methods.

An embodiment is directed to a method of analyzing and/or counting nucleic acids from single cells comprising (a) providing a sample comprising a cell within a plurality of cells, wherein the cell comprises a plurality of sample nucleic acids; (b) generating a plurality of barcoded polynucleotides from the plurality of sample nucleic acids of said cell, wherein the barcoded polynucleotide comprises a barcode sequence configured to distinguish said sample nucleic acid from other sample nucleic acids in other cells; and a sample sequence from the sample nucleic acid in the cell, wherein said sample sequence comprising a distinguishable sequence from other sample sequences of other sample nucleic acids in said cell; (c) sequencing said barcoded polynucleotide to determine the sample sequence and the barcode sequence; (d) analyzing and/or counting sample nucleic acids in said cell with said barcode sequence and sample sequence information. In some embodiments, the method further comprises generating a plurality of compartments wherein the cells are sequestered individually in the compartments prior to step (b) or in step (b). In some embodiments, the method further comprises amplifying said barcoded polynucleotide to generate a plurality of amplified barcoded polynucleotides prior to step (c). In some embodiments, the compartments comprise a form of droplet, an emulsion droplet, a liposome, a microwell, a well, a microarray, an open array, a microtiter plate, or a combination thereof. In some embodiments, the sample nucleic acids are selected from the group consisting of a total DNA, a portion of DNA, a total RNA, a portion of RNA and a combination thereof in said cell. In some embodiments, the plurality of barcoded polynucleotides are generated through a reaction selected from a group consisting of ligation, hybridization, strand transfer reaction, transposition, tagmentation, primer extension, reverse transcription, amplification, and a combination thereof. In some embodiments, the sample nucleic acids in the cell are pretreated in situ for reverse transcription, transposition, tagmentation, strand transfer reaction, ligation, hybridization, restriction enzyme digestion, crosslinking, fixation, or a combination thereof before step (b). In some embodiments, the sample sequence with the distinguishable sequence is generated by strand transfer, transposition, tagmentation, random priming, random reverse transcription, random digestion, or a combination thereof. In some embodiments, the sample sequence with the distinguishable sequence is used as a unique molecular identifier for the sample nucleic acid. In some embodiments, at least 80 percent of said sample sequences with the distinguishable sequence comprise a unique sequence different from other sample sequences in said cell. In some embodiments, at least 90 percent of said sample sequences with the distinguishable sequence comprise a unique sequence different from other sample sequences in said cell. In some embodiments, step (d) further comprises using said barcode sequence to identify a cellular origin of the sample nucleic acid and using said sample sequence to determine a uniqueness of the sample nucleic acid from other sample nucleic acids in the cell. In some embodiments, the cells consist essentially nuclei isolated from the cells.

An embodiment is directed to a method of generating barcoded polynucleotides based on DNA or RNA of a cell comprising (a) providing a sample comprising a plurality of cells, wherein the cell comprises a plurality of sample DNA or sample RNA; (b) generating a plurality of first barcoded polynucleotides from the plurality of sample DNA and a plurality of second barcoded polynucleotides from the plurality of sample RNA of said cell, wherein the first barcoded polynucleotide from sample DNA comprises: a sample sequence from the sample DNA in the cell; a barcode sequence configured to distinguish said sample DNA from other sample DNA in different cells; and a sample DNA specific adapter sequence wherein said adapter sequence comprises the same first barcoded polynucleotide from said sample DNA; wherein the second barcoded polynucleotide from sample RNA comprises a sample sequence from the sample RNA in the cell; a barcode sequence configured to distinguish said sample RNA from other sample RNA in different cells; a sample RNA specific adapter sequence wherein said adapter sequence comprises the same second barcoded polynucleotide from said sample RNA; (c) sequencing said first and the second barcoded polynucleotides to determine the sample sequence and barcode sequence; (d) analyzing the sample DNA and the sample RNA in said cell with said barcode sequence and sample sequence information. In some embodiments, the method further comprises generating a plurality of compartments wherein the cells are sequestered individually in the compartments prior to step (b) or in step (b). In some embodiments, the method further comprises amplifying said first and the second barcoded polynucleotides to generate a plurality of amplified first and second barcoded polynucleotides prior to step (c). In some embodiments, the compartments comprise a form of droplet, an emulsion droplet, a liposome, a microwell, a well, a microarray, an open array, a microtiter plate, or a combination thereof. In some embodiments, the sample DNA is a total DNA, a portion of DNA or an accessible chromatin DNA of said cell. In some embodiments, the sample RNA is a total RNA, a portion of RNA or mRNA of said cell. In some embodiments, the plurality of the first and the second barcoded polynucleotides are generated through a reaction selected from the group consisting of ligation, hybridization, strand transfer reaction, transposition, tagmentation, primer extension, reverse transcription, amplification, and a combination thereof. In some embodiments, the sample DNA in the cell are pretreated in situ for strand transfer reaction, transposition, tagmentation, ligation, hybridization, restriction enzyme digestion, crosslinking, fixation, or a combination thereof before step (b). In some embodiments, the sample RNA in the cell are pretreated in situ for reverse transcription, strand transfer reaction, transposition, tagmentation, ligation, hybridization, restriction enzyme digestion, crosslinking, fixation, or a combination thereof before step (b). In some embodiments, the sample sequence from the first barcoded polynucleotide is a distinguishable sequence from other sample sequences of other sample DNA in said cell. In some embodiments, the sample sequence from the second barcoded polynucleotide is a distinguishable sequence from other sample sequences of other sample RNA in said cell. In some embodiments, the sample sequence with a distinguishable sequence is generated by strand transfer reaction, transposition, tagmentation, random priming, random reverse transcription, random digestion, or a combination thereof. In some embodiments, the sample sequence with a distinguishable sequence is used as a unique molecular identifier for the sample DNA or sample RNA. In some embodiments, at least 80 percent of said sample sequences with a distinguishable sequence comprise a unique sequence different from other sample sequences in said cell. In some embodiments, at least 90 percent of said sample sequences with a distinguishable sequence comprise a unique sequence different from other sample sequences in said cell. In some embodiments, the barcode sequences are the same between the first and the second barcoded polynucleotides in the cell. In some embodiments, step (d) further comprises using said barcode sequence to identify common cellular origin of the sample DNA or the sample RNA, and using said sample sequences to characterize said sample DNA and said sample RNA in the cell. In some embodiments, the cells consist essentially nuclei isolated from the cells.

An embodiment is directed to a method of tracking a target's origin by barcode tagging comprising (a) sequestering one or more unique barcode templates with a target in a compartment; (b) amplifying said barcode template and modifying said target wherein the modified target is configured to link a barcode template in the compartment; (c) generating a barcode tagged modified target wherein a plurality of modified targets sharing a same one or more barcode sequences presented in said compartment; and (d) removing the separation between the compartments and collecting the barcode tagged modified targets for sequencing characterization. In some embodiments, the method further comprises identifying a compartment origin of different barcode sequences presented in the same compartment based on a shared compartment content. In some embodiments, the target is selected from the group consisting of a nucleic acid, a protein, a protein complex, a protein and nucleic acid complex, a ligand, a chemical compound, a nucleus, a cell, a microbe, a small molecule, a macromolecule, a particle, a microparticle, and a combination thereof. In some embodiments, the modification for a target is selected from the group consisting of strand transfer reaction, transposition, tagmentation, reverse transcription, amplification, primer extension, restriction enzyme digestion, hybridization, ligation, fragmentation, crosslinking, and a combination thereof. In some embodiments, the target is subject to a treatment and/or a modification before sequestering, wherein the treatment is selected from the group consisting of denaturation, permeabilization, fixation, labeling, antibody conjugation, in situ reaction, and a combination thereof; and wherein the modification is selected from the group consisting of strand transfer reaction, transposition, tagmentation, reverse transcription, amplification, primer extension, restriction enzyme digestion, hybridization, ligation, fragmentation, crosslinking, and a combination thereof. In some embodiments, sequestering compartment is selected from the group consisting of a droplet, an emulsion droplet, a liposome, a microwell, an open array, a microtiter plate, and a combination thereof. In some embodiments, the barcode template comprises a barcode sequence and at least one handle sequence configured to be used as a priming site, a hybridization site or a binding site. In some embodiments, the barcode template is a DNA, a RNA, or a DNA/RNA hybrid and said barcode sequence comprises a range from about 5 bases to about 100 bases. In some embodiments, the method of generating the barcode tagged modified target is through amplification, hybridization, primer extension, ligation, strand transfer reaction, transposition, tagmentation, or a combination thereof. In some embodiments, the target being analyzed is selected from the group consisting of a single cell, a chemical compound, a nucleic acid, a protein, a microbiome, and a combination thereof.

Although the invention has been explained with respect to an embodiment, it is to be understood that many other possible modifications and variations can be made without departing from the spirit and scope of the invention as herein described.

Further, in general with regard to the processes, systems, methods, etc. described herein, it should be understood that, although the steps of such processes, etc. have been described as occurring according to a certain ordered sequence, such processes could be practiced with the described steps performed in an order other than the order described herein. It further should be understood that certain steps could be performed simultaneously, that other steps could be added, or that certain steps described herein could be omitted. In other words, the descriptions of processes herein are provided for the purpose of illustrating certain embodiments and should in no way be construed so as to limit the claimed invention.

Moreover, it is to be understood that the above description is intended to be illustrative and not restrictive. Many embodiments and applications other than the examples provided would be apparent to those of skill in the art upon reading the above description. The scope of the invention should be determined, not with reference to the above description, but should instead be determined with reference to the appended claims, along with the full scope of equivalents to which such claims are entitled. It is anticipated and intended that future developments will occur in the arts discussed herein, and that the disclosed systems and methods will be incorporated into such future embodiments. In sum, it should be understood that the invention is capable of modification and variation and is limited only by the following claims.

Lastly, all defined terms used in the application are intended to be given their broadest reasonable constructions consistent with the definitions provided herein. All undefined terms used in the claims are intended to be given their broadest reasonable constructions consistent with their ordinary meanings as understood by those skilled in the art unless an explicit indication to the contrary is made herein. In particular, use of the singular articles such as “a,” “the,” “said,” etc. should be read to recite one or more of the indicated elements unless a claim recites an explicit limitation to the contrary.

### EXAMPLES

### Example 1. Barcoding Long Fragments in Droplets to Generate Linked Reads

This example describes a method of barcoding DNA fragments in droplets to generate linked reads.

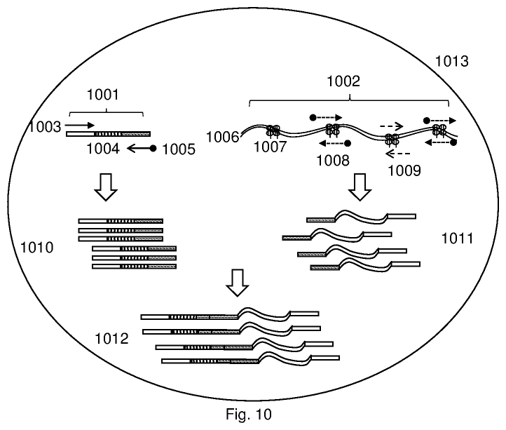

1 ng E. coli DH10b genomic DNA (FIG. 10, 1006) was strand-transferred by incubating with a wild type transpososome and a mutant MuA transpososome (1007) simultaneously using 1 μL of Barcoding Enyzme (wild type MuA transpososome) and 1 μL Tagging Enzyme (mutant MuA transpososome) from TELL-Seq WGS Library Reagent Box 1 (Universal Sequencing Technology, Carlsbad, CA) in 1× Reaction Buffer with Cofactor in 20 μL reaction volume at 37° C. for 15 minutes to form strand transfer complexes (STC, 1002). Take 1 μL of STC reaction mixture into 10 μL of an amplification aqueous solution containing 1× Pfu polymerase buffer, dNTPs, barcode templates Code 1.2 (5′-CAAGCAGAAGACGGCATACGAGATNNNatNNNNcaNNNNcgNNNTGGTCATGTGGAG ACGCTGGGACAG-3′, 1001, SEQ ID NO: 1), primers [P7 (5′-CAAGCAGAAGACGGCATACGAGAT-3′, 1003, SEQ ID NO: 2), T25 (5′-CTGTCCCAGCGTCTCCACATGACCA-3′, 1004, SEQ ID NO: 3), tsMU (5′-GCTGGGACAGGTCACTTTTCGTGCGCCGCTTCA-3′, 1008, SEQ ID NO: 4), Bio-mP5 (5′-Biotin-ACACTCTTTCCCTACATTAACTGCA-3′, 1009, SEQ ID NO: 5)] and Pfu DNA polymerase in a 0.2 mL PCR tube. Add 90 μL of 7% Abil EM90 (Evonik Corporation, Richmond, VA) in mineral oil (Sigma-Aldrich, St. Louis, MO). Set a P200 pipette at 70 L and mix the solution by pipetting up and down for 30 times in 30 seconds. Transfer 50 L mixture into another 0.2 mL PCR tube and add 50 μL of 7% Abil EM90 in mineral oil. Mix the solution by pipetting up and down for 15 times in 15 seconds. Perform amplification as following: 72° C. for 2 minutes, 94° C. for 30 seconds, 21 cycles of (94° C. for 20 seconds, 55° C. for 1 minute, 72° C. for 1 minute), 12 cycles of (94° C. for 30 seconds, 35° C. for 1 minute, 72° C. for 2 min), 72° C. for 3 minutes, hold at 4° C. At the end of PCR, add 100 μL of breaking buffer (100 mM NaCl, 10 mM Tris-HCl, pH 7.5, 0.2% SDS, 15% Isopropanol) and incubate for 10 minutes at room temperature. Spin the tube at 5,000 g for 10 minutes to separate oil and aqueous solution. Remove oil from the top layer. Transfer 70 μL of aqueous solution into a 0.5 mL low bind DNA tube and add 35 L MyOne™ Streptavidin T1 beads (Life Technologies, Carlsbad, CA) in the binding buffer. Incubate at room temperature for 15 minutes with rotation. Wash the beads three times with bead wash buffer. Resuspend the beads in 15 μL of 0.02% Tween-20. Use 5 L beads for PCR amplification in 40 μL total volume using Pfu DNA polymerase with P7 primer and one of multiplex primers from TELL-Seq Library Multiplex Primer (1-8) kit (Universal Sequencing Technology, Carlsbad, CA). Perform PCR amplification as following: 94° C. for 30 seconds, 6 cycles of (94° C. for 20 seconds, 58° C. for 1 minute, 72° C. for 1 minute), 72° C. for 3 minutes, hold at 4° C. After PCR amplification, clean up the library product with 0.9× AMPure XP beads and quantitate for sequencing. Different ratio of barcode template molecule to emulsion droplet was tested. 3 to 1 ratio was used in the example to make sure approximately 95% droplets containing at least one barcode template.

The library was sequenced in a 2×74 paired end run on a MiSeq system. The barcode templates used in the experiment contained 20-base barcode sequences and was sequenced as Index 1 read. Table 1 showed summary of the sequencing run. The mapping rates of read 1 and read 2 were 98.6% and 97.0%, respectively. Total 1,392,842 barcodes were identified.

TABLE 1
Sequencing Statistics on the E. coli library from
a 2 × 74 paired end MiSeq run

| Sequencing Metrics | Results |
|---|---|
| read_type | PE |
| read_length | 74 |
| reads_total | 7,921,891 |
| duplication rate | 17.81% |
| read1_reads_mapped_percentage | 98.6% |
| read2_reads_mapped_percentage | 97.0% |
| barcode_with_single_read | 316,297 |
| barcode_with_multi_reads | 1,281,011 |
| reads_related_to_barcode_with_multi_reads | 7,605,594 |
| barcode_corrected | 199,968 |
| error_barcode_number | 4,498 |
| final_correct_barcode_number | 1,392,842 |
| final_reads_number | 7,916,620 |

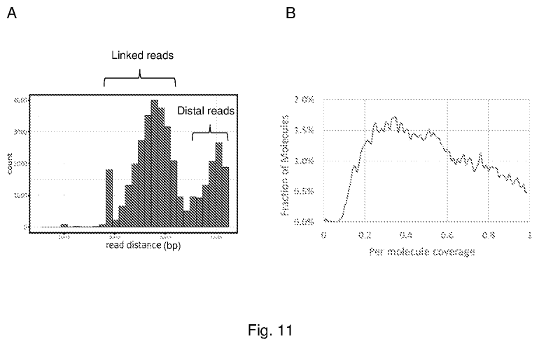

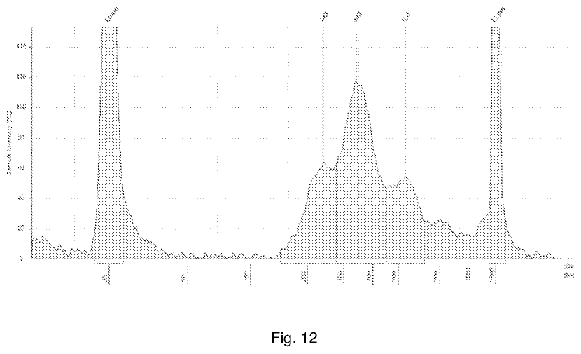

To examine if the barcoding reaction was clonal to the fragment tagged, we generated a read distance plot (FIG. 11A) which was a Read 1 read count histogram of next alignment read distance for those R1 reads sharing the same barcode sequence. If the barcoding reaction was indeed clonal to the tagged fragment, there would be many same barcoded reads with short distance (less than 50 Kb usually) next to each other which would show as the linked reads population; while the same barcoded reads arising from different genomic DNA fragments would show much large distance (greater than 100 Kb usually) in the distal reads population. FIG. 11A showed very good clonal barcoding reaction for this E. coli library. We further de novo assembled these linked reads using TuringAssembler, which was a linked read assembler and got N50 contig size of 4,591,903 bp which was very close to the full size of an E. coli DH10B genome (4,686,137 bp) with very good assembly accuracy (Table 2).

TABLE 2
QUAST results of de novo assembly
using TuringAssembler compared with
E. coli DH10B genome reference (4,686,137 bp)
de novo assembly results

| | |
|---|---|
| Genome fraction (%) | 99.054 |
| Duplication ratio | 1.068 |
| Largest alignment | 3,339,054 |
| Total aligned length | 4,950,726 |
| NG50 | 4,591,903 |
| NA50 | 3,339,054 |
| LG50 | 1 |
| LA50 | 1 |
| # mismatches per 100 kbp | 11.29 |
| # indels per 100 kbp | 1.53 |
| # N's per 100 kbp | 60.5 |

Statistics without reference

| | |
|---|---|
| # contigs | 159 |
| # contigs (>=0 bp) | 159 |
| # contigs (>=1000 bp) | 159 |
| # contigs (>=5000 bp) | 2 |
| # contigs (>=50000 bp) | 2 |
| Largest contig | 4,591,903 |
| Total length | 5,069,768 |
| Total length (>=0 bp) | 5,069,768 |
| Total length (>=1000 bp) | 5,069,768 |
| Total length (>=5000 bp) | 4,713,528 |
| Total length (>=50000 bp) | 4,713,528 |
| N50 | 4,591,903 |
| L50 | 1 |
| GC (%) | 50.71 |

### Example 2. Single Cell ATAC-Seq

Approximately 1 million PBMC cells were added to a 1.5 mL protein low-bind centrifuge tube and centrifuged at 300×g for 3 minutes. The supernatant was removed, and the pellet was resuspended in 1 mL of 1× PBS. The cells were then centrifuged again at 300×g for 3 minutes. The cell pellet was resuspended in 150 μL ice-cold lysis buffer (10 mM NaCl, 10 mM Tris pH 7.4, 3 mM MgCl₂, 0.01% digitonin, 0.1% tween, and 0.1% NP40). The cells were mixed 5× with a P200 pipette set to 100 μL and placed on ice for 3 minutes. After the 3-minute incubation, the cells were mixed 10 times with the pipette set at 100 μL. 850 μL of wash buffer (10 mM NaCl, 10 mM Tris pH 7.4, 3 mM MgCl₂, 0.1% tween) was added and mixed 5 times with a P1000 pipette set at 850 μL. The nuclei were centrifuged at 400×g for 3 minutes and resuspended in 1 mL of wash buffer. The nuclei were filtered through a 0.4 μM flowmi filter to remove any clumps and then centrifuged again at 400×g for 3 minutes. The nuclei pellet was resuspended in 20 μL of wash buffer. 2 μL of nuclei was diluted in 98 μL and counted twice to obtain an accurate cell count. The final concentration was adjusted to 25,000 nuclei/L and the nuclei were kept on ice.

5 μM Tn5ME transpososomes were assembled using EZ-Tn5™ Transposase (Lucigen, Middleton, WI) and preannealed Tn5MEDS-A and Tn5MEDS-B oligonucleotides (Picelli et al 2014). Strand transfer reaction was performed by treating 50,000 PBMC nuclei with 0.35 μM Tn5ME transpososomes in a 20 μL reaction buffer (final 10% DMF, 10 mM Tris pH7.5, and 5 mM MgCl₂, 0.33× PBS, 0.1% tween, 0.01% digitonin). The mixture was incubated on a thermal cycler for 1 hour at 37° C. After the reaction, the nuclei were diluted to a final concentration of 500 nuclei/L in nuclei resuspension buffer (10 mM NaCl, 10 mM Tris pH 7.4, 3 mM MgCl₂).

Approximately 900 tagged nuclei were used in 20 μL of amplification mix comprising Pfu DNA polymerase, dNTP, primers [Tn5-BC—R (5′-TCTCCGAGCCCACGAGAC-3′) (SEQ ID NO: 6), Tn5-R2-F28 (5′-TGGGCTCGGAGATGTGTATAAGAGACAG-3′) (SEQ ID NO: 7), P7 (5′-CAAGCAGAAGACGGCATACGAGAT-3′) (SEQ ID NO: 8), and Tn5-R1-S(5′-TCGTCGGCAGCGTCAGATGT-3′) (SEQ ID NO: 9)], barcode template Code1.3 (5′-GAAGACGGCATACGAGATNNNatNNNNcaNNNNcgNNNGTCTCGTGGGCTCGGAGA-3′) (SEQ ID NO: 10) in a 0.2 mL PCR tube. 80 μL of an oil mixture [7% Abil EM90 (Evonik Corporation, Richmond, VA) in mineral oil (Sigma-Aldrich, St. Louis, MO)] was added on top of the 20 μL amplification mixture. The targeted ratio of number of barcode templates to expected number of droplets was 3 to 1 in order to have approximately 95% of droplets containing at least one barcode template. Set a P200 pipette at 70 μL and mix the solution by pipetting up and down for 30 times in 45 seconds and additional 15 times in 30 seconds. The following PCR program was performed: 72° C. for 5 minutes, 95° C. for 30 seconds, 20 cycles of (95° C. for 15 seconds, 58° C. for 30 seconds, and 72° C. for 20 seconds), 5 cycles of (95° C. for 20 seconds, 40° C. for 2 minutes, and 72° C. for 30 seconds), 72° C. for 2 minutes, 20° C. for 1 minute, and hold at 4° C.

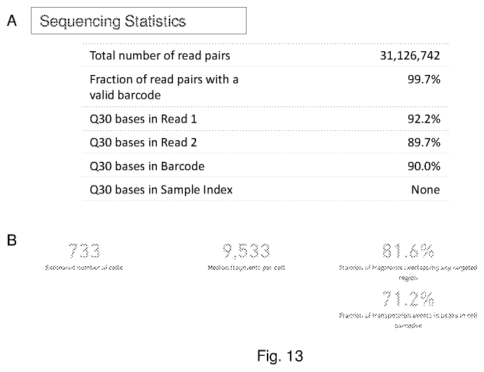

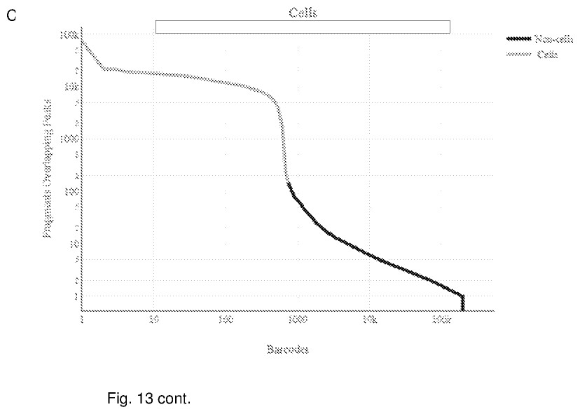

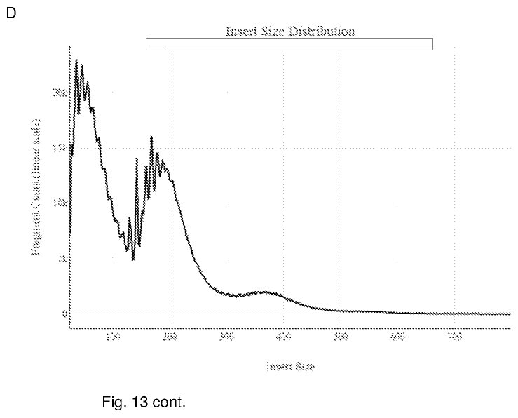

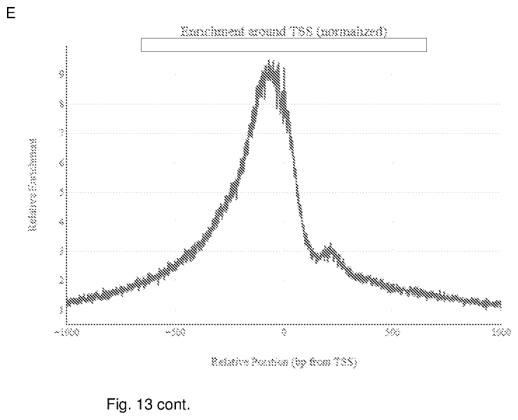

After droplet amplification, the larger droplets settle to the bottom leaving smaller droplet and oil on top. The top 50 μL was removed and discarded without disturbing bottom layer of settled droplets. 50 μL of breaking solution (100 mM NaCl, 10 mM Tris-HCl, pH 7.5, 0.2% SDS, 15% Isopropanol) was added to the emulsion and mixed 10 times. The emulsion was centrifuged for 8 minutes on a 10 k minifuge. An additional 10-15 μL of the top oil layer was removed and discarded, being sure not to remove any of the bottom aqueous layer. Slowly, 60 μL of the bottom aqueous solution was removed from the bottom and placed in a new tube, while being careful not to aspirate any residual oil on the top layer. A 1.2× bead cleanup was performed by adding 72 μL of AMPure XP beads to the aqueous solution. The mixture was incubated for 5 minutes at room temperature and then placed on a magnet for 2-3 minutes (or until clear). The clear supernatant was removed and two washes using 200 μL freshly prepared 80% Ethanol was performed. Washed beads were resuspended in 33 μL of low TE buffer. 30 μL was removed and placed into a new PCR tube. 15 μL of cleaned up products were used for a final PCR amplification in a 40 μL mix of 1× Phusion Hot Start II High Fidelity PCR master mix with P7 primer and one of multiplex primers from TELL-Seq Library Multiplex Primer (1-8) kit (Universal Sequencing Technology, Carlsbad, CA) to generate an Illumina sequencing library. The following PCR program was performed: 95° C. for 30 seconds, 5 cycles of (95° C. for 20 seconds, 63° C. for 30 seconds, and 72° C. for 30 seconds), 72° C. for 2 minutes, and hold at 4° C. A 1.2× AMPure XP bead cleanup was performed by adding 48 μL of AMPure XP beads to the PCR product. The mixture was incubated for 5 minutes at room temperature and then placed on a magnet for 2-3 minutes (or until clear). The clear supernatant was removed and two washes using 200 μL freshly prepared 80% Ethanol was performed. Washed beads were resuspended in 25 μL of low TE buffer. 23 μL was removed and transferred into a new PCR tube. The final library was quantified using a high sensitivity D1000 screen tape on a TapeStation (FIG. 12). The library was sequenced on a NextSeq 500. Before standard Cell Ranger analysis, different barcodes from the same droplet were merged based on their share fragment profile. Total 31,126,742 sequencing read pairs were produced. 99.7% reads pairs contained a valid barcode (FIG. 13A). Further analysis using Cell Ranger analysis software identified 733 cells (FIG. 13B) with 9533 median fragments per cell. Knee-plot demonstrated a clear single-cell behavior (FIG. 13C). The library insert size profile showed clear nucleosomal banding pattern (FIG. 13D) and sequencing reads showed strong enrichment around transcription start site (FIG. 13E).

### REFERENCES

Adey A. et al. 2010. Genome Biol. 11, R119.
Amini S. et al. 2014. Nature Genetics, 46(12):1343-1349.
Au, T. et al. 2004. EMBO J., 23: 3408-3420.
Buenrostro J. D. et al. 2013. Nature Methods, 10(12): 1213-1218.
Buenrostro, J. D. et al. 2015. Nature, 523: 486-490.
Burton B. M. and Baker T. A. 2003. Chemistry & Biology 10: 463-472.
Caruccio N. 2011. Methods Mol. Biol. 733: 241-255.
Kavanagh I, Kiiskinen L. L. and Haakana H. 2013. Unite State Patent Application Publication US2013/0023423.
Kurihara K. et al. 2011. Nat. Chem. 3: 775-781.
Laouini A. et al. 2012. Colloid Sci. Biotechnol. 1: 147-168.
Mizuuchi M., Baker T. A. and Mizuuchi K. 1992. Cell 70, 303-311.
Savilahti H., P. A. Rice, and K. MiZuuchi. 1995. EMBO J. 14:4893-4903.
Stoeckius M., et al. 2017. Nature Methods 14: 865-868.
Surette M., Buch S. J. and Chaconas G. 1987. Cell 70: 303-311.
Reznikoff W. S. 2008. Annual Review of Genetics 42(1): 269-286.

1. A method of analyzing and/or counting nucleic acids from single cells comprising:
a. providing a sample comprising a cell within a plurality of cells, wherein the cell comprises a plurality of sample nucleic acids;
b. generating a plurality of barcoded polynucleotides from the plurality of sample nucleic acids of said cell, wherein the barcoded polynucleotide comprises:
i. a barcode sequence configured to distinguish said sample nucleic acid from other sample nucleic acids in other cells;
ii. a sample sequence from the sample nucleic acid in the cell, wherein said sample sequence comprising a distinguishable sequence from other sample sequences of other sample nucleic acids in said cell;
c. sequencing said barcoded polynucleotide to determine the sample sequence and the barcode sequence;
d. analyzing and/or counting sample nucleic acids in said cell with said barcode sequence and sample sequence information.

2. The method of claim 1, further comprising generating a plurality of compartments wherein the cells are sequestered individually in the compartments prior to step (b) or in step (b).

3. The method of claim 1, further comprising amplifying said barcoded polynucleotide to generate a plurality of amplified barcoded polynucleotides prior to step (c).

4. The method of claim 2, wherein said compartments comprise a form of droplet, an emulsion droplet, a liposome, a microwell, a well, a microarray, an open array, a microtiter plate, or a combination thereof.

5. The method of claim 1, wherein said sample nucleic acids are selected from the group consisting of a total DNA, a portion of DNA, a total RNA, a portion of RNA and a combination thereof in said cell.

6. The method of claim 1, wherein said plurality of barcoded polynucleotides are generated through a reaction selected from a group consisting of ligation, hybridization, strand transfer reaction, transposition, tagmentation, primer extension, reverse transcription, amplification, and a combination thereof.

7. The method of claim 1, wherein said sample nucleic acids in the cell are pretreated in situ for reverse transcription, transposition, tagmentation, strand transfer reaction, ligation, hybridization, restriction enzyme digestion, crosslinking, fixation, or a combination thereof before step (b).

8. The method of claim 1, wherein said sample sequence with the distinguishable sequence is generated by strand transfer, transposition, tagmentation, random priming, random reverse transcription, random digestion, or a combination thereof.

9. The method of claim 1, wherein said sample sequence with the distinguishable sequence is used as an unique molecular identifier for the sample nucleic acid.

10. The method of claim 1, wherein at least 80 percent of said sample sequences with the distinguishable sequence comprise an unique sequence different from other sample sequences in said cell.

11. The method of claim 1, wherein at least 90 percent of said sample sequences with the distinguishable sequence comprise an unique sequence different from other sample sequences in said cell.

12. The method of claim 1, wherein step (d) further comprises using said barcode sequence to identify a cellular origin of the sample nucleic acid and using said sample sequence to determine a uniqueness of the sample nucleic acid from other sample nucleic acids in the cell.

13. The method of claim 1, wherein said cells consist essentially nuclei isolated from the cells.

14. A method of generating barcoded polynucleotides based on DNA or RNA of a cell comprising:
a. providing a sample comprising a plurality of cells, wherein the cell comprises a plurality of sample DNA or sample RNA;
b. generating a plurality of first barcoded polynucleotides from the plurality of sample DNA and a plurality of second barcoded polynucleotides from the plurality of sample RNA of said cell,
wherein the first barcoded polynucleotide from sample DNA comprises:
i. a sample sequence from the sample DNA in the cell;
ii. a barcode sequence configured to distinguish said sample DNA from other sample DNA in different cells;
iii. a sample DNA specific adapter sequence wherein said adapter sequence comprises the same first barcoded polynucleotide from said sample DNA;
wherein the second barcoded polynucleotide from sample RNA comprises:
i. a sample sequence from the sample RNA in the cell;
ii. a barcode sequence configured to distinguish said sample RNA from other sample RNA in different cells;
iii. a sample RNA specific adapter sequence wherein said adapter sequence comprises the same second barcoded polynucleotide from said sample RNA;
c. sequencing said first and the second barcoded polynucleotides to determine the sample sequence and barcode sequence;
d. analyzing the sample DNA and the sample RNA in said cell with said barcode sequence and sample sequence information.

15. The method of claim 14, further comprising generating a plurality of compartments wherein the cells are sequestered individually in the compartments prior to step (b) or in step (b).

16. The method of claim 14, further comprising amplifying said first and the second barcoded polynucleotides to generate a plurality of amplified first and second barcoded polynucleotides prior to step (c).

17. (canceled)

18. (canceled)

19. (canceled)

20. (canceled)

21. (canceled)

22. (canceled)

23. The method of claim 14, wherein said sample sequence from the first barcoded polynucleotide is a distinguishable sequence from other sample sequences of other sample DNA in said cell.

24. The method of claim 14, wherein said sample sequence from the second barcoded polynucleotide is a distinguishable sequence from other sample sequences of other sample RNA in said cell.

25. (canceled)

26. (canceled)

27. (canceled)

28. (canceled)

29. The method of claim 14, wherein said barcode sequences are the same between the first and the second barcoded polynucleotides in the cell.

30. (canceled)

31. (canceled)

32. A method of tracking a target's origin by barcode tagging comprising:
a. sequestering one or more unique barcode templates with a target in a compartment;
b. amplifying said barcode template and modifying said target wherein the modified target is configured to link a barcode template in the compartment;
c. generating a barcode tagged modified target wherein a plurality of modified targets sharing a same one or more barcode sequences presented in said compartment;
d. removing the separation between the compartments and collecting the barcode tagged modified targets for sequencing characterization.

33-41. (canceled)
